(19) United States
(12) Patent Application Publication
(10) Pub. No.: US 2021/0220037 A1
(43) Pub. Date: Jul. 22, 2021

(54) METHOD FOR REDUCTION OF CELL VIABILITY BY COLD PLASMA TREATMENT ON BREAST CANCERS BASED ON MOLECULAR PROFILING

(71) Applicant: Jerome Canady Research Institute for Advanced Biological and Technological Sciences, Takoma Park, MD (US)

(72) Inventors: Jerome Canady, Lakeland, FL (US); Lawan Ly, Annandale, VA (US); Saravana Murthy, Owings Mills, MD (US); Xiaoqian Cheng, Fairfax, VA (US); Taisen Zhuang, Rockville, MD (US)

(21) Appl. No.: 17/134,621

(22) Filed: Dec. 28, 2020

Related U.S. Application Data
(60) Provisional application No. 62/953,767, filed on Dec. 26, 2019.

(51) Int. Cl. A61B 18/04; G01N 33/574; A61B 17/00; A61B 18/00

(52) U.S. Cl. CPC A61B 18/042; G01N 33/57492; A61B 2018/00702; A61B 2018/00333; A61B 2018/00773; A61B 2017/00199

(57) ABSTRACT

A method for treatment of breast cancer using molecular profiling. The method comprises performing a molecular analysis of target breast cancer cells, identifying markers associated with the target breast cancer cells based on the performed molecular analysis, selecting on a graphical user interface of a cold atmospheric plasma generator the identified markers associated with the target breast cancer cells, selecting with a processor in the cold atmospheric plasma generator preferred cold atmospheric plasma settings associated with the identified markers in a database stored in a storage in the cold atmospheric plasma generator, and applying cold atmospheric plasma with the cold atmospheric plasma generator at the selected cold atmospheric pressure settings to the target breast cancer cells.

### CROSS-REFERENCE TO RELATED APPLICATIONS

The present application claims the benefit of the filing date of U.S. Provisional Patent Application Ser. No. 62/953,767 filed by the present inventors on Dec. 26, 2019.

The aforementioned provisional patent application is hereby incorporated by reference in its entirety.

### STATEMENT REGARDING FEDERALLY SPONSORED RESEARCH OR DEVELOPMENT

None.

### BACKGROUND OF THE INVENTION

### Field of the Invention

The present invention relates to systems and methods for treating cancer with cold atmospheric plasma.

### Brief Description of the Related Art

Among women worldwide, breast cancer is the most frequently diagnosed cancer and the most common cause of cancer death. Bray, F., et al., Global cancer statistics 2018: GLOBOCAN estimates of incidence and mortality worldwide for 36 cancers in 185 countries. CA Cancer J Clin, 2018. 68(6): p. 394-424. The major breast cancer molecular subtypes are based on estrogen receptor (ER), progesterone receptor (PR) and human epidermal growth factor receptor 2 (HER2) expression. Howlader, N., et al., Differences in Breast Cancer Survival by Molecular Subtypes in the United States. Cancer Epidemiology, Biomarkers & Prevention, 2018. 27(6): p. 619-626. With a total of eight combinations of ER, PR and HER2 expression (see Bauer, K., C. Parise, and V. Caggiano, Use of ER/PR/HER2 subtypes in conjunction with the 2007 St Gallen Consensus Statement for early breast cancer. BMC Cancer, 2010. 10), breast cancer is acknowledged as a highly complex disease. Molecular profiling, however, can provide information on disease prognosis and therapeutic approach. Kittaneh, M., A. J. Montero, and S. Gluck, Molecular profiling for breast cancer: a comprehensive review. Biomark Cancer, 2013. 5: p. 61-70; Duffy, M. J., et al., Clinical use of biomarkers in breast cancer: Updated guidelines from the European Group on Tumor Markers (EGTM). Eur J Cancer, 2017. 75: p. 284-298 N Approximately 75% of breast cancers are ER-positive (ER+) while 55-65% are PR-positive (PR+). See Anderson, W. F., et al., Estrogen receptor breast cancer phenotypes in the Surveillance, Epidemiology, and End Results database. Breast Cancer Research and Treatment, 2002. 76(1): p. 27-36; Colditz, G. A., et al., Risk factors for breast cancer according to estrogen and progesterone receptor status. J Natl Cancer Inst, 2004. 96(3): p. 218-28; and Nadji, M., et al., Immunohistochemistry of estrogen and progesterone receptors reconsidered: experience with 5,993 breast cancers. Am J Clin Pathol, 2005. 123(1): p. 21-7.

Survival rates of patients are highest with ER+/PR+ tumors, intermediate with either ER+/PR− or ER−/PR+ tumors, and lowest with ER−/PR− tumors. Alanko, A., et al., Significance of Estrogen and Progesterone Receptors, Disease-Free Interval, and Site of First Metastasis on Survival of Breast Cancer Patients. Cancer, 1985. 56(7): p. 1696-700

Several studies have reported changes in hormone receptor status between primary and metastatic breast cancer with discordance rates estimated to be 20% for ER and 40% for PR (both of which are higher than HER2 discordance rate). See, Curtit, E., et al., Discordances in estrogen receptor status, progesterone receptor status, and HER2 status between primary breast cancer and metastasis. Oncologist, 2013. 18(6): p. 667-74; RJ, B., et al., Changes in Estrogen Receptor, Progesterone Receptor and Her-2/neu Status with Time: Discordance Rates Between Primary and Metastatic Breast Cancer. Anticancer Research, 2009. 29(5): p. 1557-62; Sighoko, D., et al., Discordance in hormone receptor status among primary, metastatic, and second primary breast cancers: biological difference or misclassification? Oncologist, 2014. 19(6): p. 592-601; and Liedtke, C., et al., Prognostic impact of discordance between triple-receptor measurements in primary and recurrent breast cancer. Ann Oncol, 2009. 20(12): p. 1953-8. Patients with discordant receptor status have lower rates of survival than patients with consistent receptor status possibly due ineffective therapeutic interventions compared to patients with consistent receptor status. Tamoxifen, a selective estrogen-receptor modulator, reduces risk of disease recurrence by 47% after 5 years and mortality by 26% after 10 years in ER+ patients (Riggs, B. L. and L. C. Hartmann, Selective estrogen-receptor modulators—mechanisms of action and application to clinical practice. N Engl J Med, 2003. 348(7): p. 618-29) but increases the risk for thromboembolic events significantly (Pritchard, K. I., et al., Increased Thromboembolic Complications with Concurrent Tamoxifen and Chemotherapy in a Randomized Trial of Adjuvant Therapy for Women with Breast Cancer. Journal of Clinical Oncology, 1996. 14(10): p. 2731-7) and absence of ER expression is associated with de novo resistance to tamoxifen (Johnston, S. R. D., et al., Changes in Estrogen Receptor, Progesterone Receptor, and pS2 Expression in Tamoxifen-resistant Human Breast Cancer. Cancer Research, 1995. 55(15): p. 3331-8). In comparison, patients treated with letrozole, an aromatase inhibitor, had a lower chance of relapse over a 5-year period but reported increased incidences of adverse events. Coates, A. S., et al., Five years of letrozole compared with tamoxifen as initial adjuvant therapy for postmenopausal women with endocrine-responsive early breast cancer: update of study BIG 1-98. J Clin Oncol, 2007. 25(5): p. 486-92.

HER2 amplification (HER2+) occurs in approximately 25-30% of primary human breast cancers and is the most significant prognostic factor compared to other factors such as ER, PR, tumor size, and age. See, Slamon, D. J., et al., Human breast cancer: correlation of relapse and survival with amplification of the HER-2/neu oncogene. Science, 1987. 235(4785): p. 177-82; and Slamon, D. J., et al., Studies of the HER-2/neu proto-oncogene in human breast and ovarian cancer. Science, 1989. 244(4905): p. 707-12. HER2, in addition to other human epidermal growth factor (hEGF) receptors, is involved in a complex network of pathways that are responsible for signaling normal cellular processes such as cell growth, migration, differentiation, and death. Yarden, Y. and M. X. Sliwkowski, Untangling the ErbB signaling network. Nature Reviews Molecular Cell Biology, 2001. 2(2): p. pages 127-137. An overexpression of HER2, therefore, promotes aggressive tumor behavior which is characterized by significantly decreased rates of disease-free and overall survival. Trastuzumab (Herceptin), a humanized monoclonal antibody developed to target and inhibit the function of HER2 (Molina, M. A., et al., Trastuzumab (Herceptin), a Humanized Anti-HER2 Receptor Monoclonal Antibody, Inhibits Basal and Activated HER2 Ectodomain Cleavage in Breast Cancer Cells. Cancer Research, 2001. 61(12): p. 4744-9), is a generally well-tolerated monotherapy for metastatic HER2-positive breast cancers. See, Vogel, C. L., et al., Efficacy and Safety of Trastuzumab as a Single Agent in First-Line Treatment of HER2-Overexpressing Metastatic Breast Cancer. Journal of Clinical Oncology, 2002. 20(3): p. 719-726; and Baselga, J., et al., Phase II study of efficacy, safety, and pharmacokinetics of trastuzumab monotherapy administered on a 3-weekly schedule. J Clin Oncol, 2005. 23(10): p. 2162-71. Randomized trials have reported adjuvant and neoadjuvant trastuzumab improved chance of overall survival in HER2+ breast cancer patients than those who received only chemotherapy. See, Smith, I., et al., 2-year follow-up of trastuzumab after adjuvant chemotherapy in HER2 positive breast cancer: a randomised controlled trial. The Lancet, 2007. 369(9555): p. 29-36; Gianni, L., et al., Neoadjuvant and adjuvant trastuzumab in patients with HER2-positive locally advanced breast cancer (NOAH): follow-up of a randomised controlled superiority trial with a parallel HER2-negative cohort. The Lancet Oncology, 2014. 15(6): p. 640-647; and Slamon, D. J., et al., Use of Chemotherapy plus a Monoclonal Antibody against HER2 for Metastatic Breast Cancer That Overexpresses HER2. New England Journal of Medicine, 2001. 344(11): p. 783-792. However, risk of cardiac dysfunction was significantly raised in combination with anthracycline and cyclophosphamide according to Slamon et. al. Moreover, a 25-35% chance of central nervous system metastasis 6-12 months after the start of trastuzumab-based therapies due to the inability of trastuzumab to cross the blood-brain barrier have been reported. See, Clayton, A., et al., Incidence of cerebral metastases in patients treated with trastuzumab for metastatic breast cancer. British Journal of Cancer, 2004. 91: p. 639-643; Bendell, J. C., et al., Central nervous system metastases in women who receive trastuzumab-based therapy for metastatic breast carcinoma. Cancer, 2003. 97(12): p. 2972-7; and Gori, S., et al., Central nervous system metastases in HER-2 positive metastatic breast cancer patients treated with trastuzumab: incidence, survival, and risk factors. Oncologist, 2007. 12(7): p. 766-73. FDA-approved dual anti-HER2 regimen, pertuzumab in combination with trastuzumab and docetaxel, significantly improved progression-free survival but with >30% of patients exhibiting side effects such as diarrhea, neutropenia, nausea, fatigue, and peripheral neuropathy. Blumenthal, G. M., et al., First FDA approval of dual anti-HER2 regimen: pertuzumab in combination with trastuzumab and docetaxel for HER2 positive metastatic breast cancer. Clin Cancer Res, 2013. 19(18): p. 4911-6.

The overexpression of ER, PR, and HER2 is classified as triple positive breast cancer (TPBC). Vici, P., et al., Triple positive breast cancer: a distinct subtype? Cancer Treat Rev, 2015. 41(2): p. 69-76 It is estimated that 10% of all breast cancer tumors are ER+/PR+/HER2+. Howlader, N., et al., US incidence of breast cancer subtypes defined by joint hormone receptor and HER2 status. J Natl Cancer Inst, 2014. 106(5).; Onitilo, A. A., et al., Breast cancer subtypes based on ER/PR and Her2 expression: comparison of clinicopathologic features and survival. Clin Med Res, 2009. 7(1-2): p. 4-13. Since HR and HER2 receptors are expressed, TPBCs can be treated with hormonal and HER2− targeted therapies. Overall and disease-free survival in ER+/PR+/HER2+ patients significantly improves in response to a combination of endocrine therapy, trastuzumab, and chemotherapy. Hayashi, N., et al., Adding hormonal therapy to chemotherapy and trastuzumab improves prognosis in patients with hormone receptor-positive and human epidermal growth factor receptor 2-positive primary breast cancer. Breast Cancer Res Treat, 2013. 137(2): p. 523-31. However, endocrine therapy resistance has been linked to crosstalk between ER and HER2 signaling pathways. See, AlFakeeh, A. and C. Brezden-Masley, Overcoming endocrine resistance in hormone receptor-positive breast cancer. Curr Oncol, 2018. 25(Suppl 1): p. S18-S27; Osborne, C. K. and R. Schiff, Mechanisms of endocrine resistance in breast cancer. Annu Rev Med, 2011. 62: p. 233-47; and Giuliano, M., M. V. Trivedi, and R. Schiff, Bidirectional Crosstalk between the Estrogen Receptor and Human Epidermal Growth Factor Receptor 2 Signaling Pathways in Breast Cancer: Molecular Basis and Clinical Implications. Breast Care (Basel), 2013. 8(4): p. 256-62.

Triple negative breast cancer (TNBC) characterized by the lack of expression of ER, PR, and HER2 accounts for approximately 10 to 20% of all breast cancers. Trivers, K. F., et al., The epidemiology of triple-negative breast cancer, including race. Cancer Causes Control, 2009. 20(7): p. 1071-82; Rakha, E. A., et al., Prognostic markers in triple-negative breast cancer. Cancer, 2007. 109(1): p. 25-32; Qiu, J., et al., Comparison of Clinicopathological Features and Prognosis in Triple-Negative and Non-Triple Negative Breast Cancer. J Cancer, 2016. 7(2): p. 167-73; Dent, R., et al., Triple-negative breast cancer: clinical features and patterns of recurrence. Clin Cancer Res, 2007. 13(15 Pt 1): p. 4429-34. Compared to all other breast cancer phenotypes, despite adjuvant chemotherapy TNBC has a significantly higher the rate of recurrence and risk of metastatic spread to the lungs, liver, and brain. Dent, R., et al., Triple-negative breast cancer: clinical features and patterns of recurrence. Clin Cancer Res, 2007. 13(15 Pt 1): p. 4429-34. While neoadjuvant chemotherapy has achieved a higher rate of pathologic complete response in TNBC patients than in non-TNBC patients, TNBC patients with residual disease have higher recurrence and death rates in the first 3 years than non-TNBC patients with residual disease. Liedtke, C., et al., Response to Neoadjuvant Therapy and Long-Term Survival in Patients with Triple-Negative Breast Cancer. Journal of Clinical Oncology, 2008. 26(8): p. 1275-1281. TNBC patients do not respond to endocrine therapy or HER2-targeted therapies, such as trastuzumab. Liedtke, C., et al., Response to neoadjuvant therapy and long-term survival in patients with triple-negative breast cancer. Journal of Clinical Oncology, 2008. 26(8): p. 1275-81; Wahba, H. A. and H. A. El-Hadaad, Current approaches in treatment of triple-negative breast cancer. Cancer Biol Med, 2015. 12(2): p. 106-16. Recently there has been a growing interest in TRAIL (TNF (tumor necrosis factor)-related apoptosis-inducing ligand) which activates Death Receptors (DR) 4 and 5 to induce apoptosis. Oakman, C., G. Viale, and A. Di Leo, Management of triple negative breast cancer. Breast, 2010. 19(5): p. 312-21. Potential TRAIL-targeting therapies have demonstrated the ability to induce apoptosis in TNBC cell lines with a mesenchymal phenotype (Rahman, M., et al., TRAIL induces apoptosis in triple-negative breast cancer cells with a mesenchymal phenotype. Breast Cancer Res Treat, 2009. 113(2): p. 217-30) and suppresses tumor growth and metastasis in mice models. Jyotsana, N., et al., Minimal dosing of leukocyte targeting TRAIL decreases triple-negative breast cancer metastasis following tumor resection. Science Advances, 2019. 5(7); Greer, Y. E., et al., MEDI3039, a novel highly potent tumor necrosis factor (TNF)-related apoptosis-inducing ligand (TRAIL) receptor 2 agonist, causes regression of orthotopic tumors and inhibits outgrowth of metastatic triple-negative breast cancer. Breast Cancer Res, 2019. 21(1): p. 27. Another target of interest for TNBC are cyclin dependent kinases (CDK) or cell cycle regulators. Lynce, F., A. N. Shajahan-Haq, and S. M. Swain, CDK4/6 inhibitors in breast cancer therapy: Current practice and future opportunities. Pharmacol Ther, 2018. 191: p. 65-73. Several FDA-approved CDK4/6 inhibitors (palbociclib, ribociclib and abemaciclib) have been shown to improve survivability, although accompanied by neutropenia, fatigue, nausea and diarrhea. Up until recently, TNBC treatment options were limited to surgery, chemotherapy, and radiotherapy, however, the development of therapies non-dependent on receptor status is promising for TNBC patients.

Cold atmospheric plasma (CAP) technology utilizes ionized gas for various applications such as wound healing, HIV treatment, and cancer treatment. Keidar, M., et al., Cold atmospheric plasma in cancer therapy. Physics of Plasmas, 2013. 20(5); Arpitha, P., Cold Atmospheric Plasma as an Alternative Therapy for Cancer Treatment. Cell & Developmental Biology, 2015. 04(02). A Cold Plasma Conversion System, composed of the Cold Plasma Scalpel with a Cold Plasma Conversion Unit, is an electrosurgical system that produces CAP for the treatment of surgical margins upon tumor resection (U.S. Pat. No. 9,999,462). One of the advantages of cold atmospheric plasma systems is that the CAP temperature remains between 26-30° C. during the duration of the treatment (Cheng, X., et al., Treatment of Triple-Negative Breast Cancer Cells with the Canady Cold Plasma Conversion System: Preliminary Results. Plasma, 2018. 1(1): p. 218-228) and does not cause any thermal or physical damage to normal tissue (Ly, L., et al., A New Cold Plasma Jet: Performance Evaluation of Cold Plasma, Hybrid Plasma and Argon Plasma Coagulation. Plasma, 2018. 1(1): p. 189-200). Our previous studies have demonstrated the ability of the system to significantly reduce the viability of various malignant solid tumor cell lines (including pancreatic adenocarcinoma, renal adenocarcinoma, esophageal adenocarcinoma, colorectal carcinoma, and ovarian adenocarcinoma) by 80-99% 48 hours post-CAP treatment. Rowe, W., et al., The Canady Helios Cold Plasma Scalpel Significantly Decreases Viability in Malignant Solid Tumor Cells in a Dose Dependent Manner. Plasma, 2018. 1(1): p. 177-188. For breast adenocarcinoma, TNBC in particular, an 80% reduction of cell viability was achieved 48 hours after treatment with the CCPCS.

Several different systems and methods for performing Cold Atmospheric Plasma (CAP) treatment have been disclosed. For example, U.S. Pat. No. 10,213,614 discloses a two-electrode system for CAP treatment. U.S. Pat. Nos. 9,999,462 and 10,023,858 each disclose a converter unit for using a traditional electrosurgical system with a single electrode CAP accessory to perform CAP treatment. WO 2018191265A1 disclosed an integrated electrosurgical generator and gas control module for performing CAP.

### SUMMARY OF THE INVENTION

In a preferred embodiment, the present invention is a novel treatment approach for cancer using Cold Atmospheric Plasma. More specifically, the present invention is a for reduction of cell viability by cold plasma conversion system treatment on breast cancers based on molecular profiling.

In a preferred embodiment, the present invention is a method for treatment of breast cancer. The method comprises performing a molecular analysis of target breast cancer cells, identifying markers associated with the target breast cancer cells based on the performed molecular analysis, selecting on a graphical user interface of a cold atmospheric plasma generator the identified markers associated with the target breast cancer cells, selecting with a processor in the cold atmospheric plasma generator preferred cold atmospheric plasma settings associated with the identified markers in a database stored in a storage in the cold atmospheric plasma generator, and applying cold atmospheric plasma with the cold atmospheric plasma generator at the selected cold atmospheric pressure settings to the target breast cancer cells. The selected CAP settings may include time and power. The method may further comprise treating the target breast cancer cells with one of chemotherapy and radiation therapy to reduce resistance of the target cancer cells' resistance to cold atmospheric plasma treatment.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature, and not as restrictive. Additional objects and advantages of the invention will be set forth in part in the description which follows and in part will be obvious from the description or may be learned by practice of the invention.

### BRIEF DESCRIPTION OF THE DRAWINGS

For a more complete understanding of the present invention and the advantages thereof, reference is now made to the following description and the accompanying drawings, in which:

FIG. 1 is a flow diagram illustrating a method in accordance with a preferred embodiment of the present invention.

FIG. 2 is a perspective view of a preferred embodiment of a gas-enhanced electrosurgical generator that may be used in a preferred embodiment of the present invention.

FIG. 3 is a block diagram of a cold atmospheric plasma generator in accordance with a preferred embodiment of the present invention.

FIG. 4A is a block diagram of an embodiment of a cold atmospheric plasma system with an electrosurgical generator and a low frequency converter for producing cold plasma.

FIG. 4B is a block diagram of an embodiment of an integrated cold atmospheric plasma system that can perform multiple types of plasma surgeries.

FIG. 5 is perspective view of a cold atmospheric plasma probe that may be used in a preferred embodiment of the present invention.

FIG. 6A is an assembly view of a handpiece of a cold atmospheric plasma probe that may be used in a preferred embodiment of the present invention.

FIG. 6B is an assembly view of a cable harness of a cold atmospheric plasma probe that may be used in a preferred embodiment of the present invention.

FIGS. 7A, 7B and 7C are bar graphs showing the viability of CAP treated cells in relative percentage to no treatment controls among all 7 tested breast cancer cell lines at 80 P (FIG. 7A), 100 P (FIG. 7B), and 120 P (FIG. 7C) after 48 hours. CAP treatment significantly reduces viability of all tested breast cancer cell lines. * p≤0.05.

FIGS. 8A and 8B illustrate comparisons of the reduction of viability between cell lines. FIG. 8A is a chart showing whether there is statistical difference between two cell lines under specific CAP treatments (p≤0.05). FIG. 8B is a chart showing percentage of CAP conditions in which there is a statistical difference between two cell lines (p≤0.05).

FIG. 9A is a bar graph showing the reduction of cell viability of ER+/PR+/HER2− cell lines 48 hours after CAP treatment compared to no treatment controls. CAP treatment significantly reduces viability of MCF-7 and T-47D at all tested doses. * p≤0.05.

FIG. 9B is a bar graph showing the reduction of cell viability of an ER−/PR−/HER2+ cell line 48 h after CAP treatment compared to no treatment controls. CAP treatment significantly reduces viability of Sk-Br-3 at all tested doses. * p≤0.05.

FIG. 9C is a bar graph showing the reduction of cell viability of an ER+/PR+/HER2+ cell line 48 h after CAP treatment compared to no treatment controls. CAP treatment significantly reduces viability of BT-474 at nearly all tested doses. * p≤0.05.

FIG. 9D is a bar graph showing the reduction of cell viability of ER−/PR−/HER2− cell lines 48 h after CAP treatment compared to no treatment controls.

FIG. 10 is a bar graph further illustrating reduction of MDA-MB-231 cell viability by CCPCS 48 h after CAP treatment compared to no treatment controls.

FIGS. 11A-11B are bar graphs showing (FIG. 11A) treatment duration and (FIG. 11B) power as factors in the reduction of MCF-7 viability compared to no treatment controls.

FIGS. 12A and 12B are bar graphs showing (FIG. 12A) treatment duration and (FIG. 12B) power as factors in the reduction of T-47D viability compared to no treatment controls.

FIGS. 13A and 13B are bar graphs showing (FIG. 13A) treatment duration and (FIG. 13B) power as factors in the reduction of SK-BR-3 viability compared to no treatment controls.

FIGS. 14A and 14B are bar graphs showing (FIG. 14A) treatment duration and (FIG. 14B) power as factors in the reduction of BT-474 viability compared to no treatment controls.

FIGS. 15A and 15B are bar graphs showing (FIG. 15A) treatment duration and (FIG. 15B) power as factors in the reduction of MDA-MB-231 viability compared to no treatment controls.

FIGS. 16A and 16B are bar graphs showing (FIG. 16A) treatment duration and (FIG. 16B) power as factors in the reduction of Hs578T viability compared to no treatment controls.

FIGS. 17A and 17B are bar graphs showing (FIG. 17A) treatment duration and (FIG. 17B) power as factors in the reduction of HCC1806 viability compared to no treatment controls.

### DETAILED DESCRIPTION OF THE PREFERRED EMBODIMENTS

Breast cancer is a heterogenous disease which can be classified into subtypes by the presence or absence of estrogen receptor (ER), progesterone receptor (PR), and human epidermal growth factor 2 receptor (HER2). Cold atmospheric plasma (CAP) has been shown to be a potential treatment for cancer. With the present invention, a 92-99% reduction of viability by CAP treatment was achieved across all tested breast cancer cell lines (p 0.05). Increasing treatment duration and power significantly reduced breast cancer cell viability (** f_{(2,2)}≤0.0176, *** f_{(5,14)}≤0.0033). The present invention utilizes the discovery that CAP sensitivity in breast cancer cells is based on receptor status. Cells with identical receptor status show the least difference in CAP sensitivity (p≤0.05), the difference being 33% between the two ER+/PR+/HER2− cell lines (p≤0.05) and 22-44% between the three TNBC cell lines (p≤0.05). HER2-negative status, irrespective of ER/PR status, also showed ≤50% difference in CAP sensitivity (p≤0.05). Moreover, demonstration of ER−/PR−/HER2+ CAP susceptibility and ER+/PR+/HER2+ CAP resistance suggests that ER/PR status is a significant factor in determining CAP sensitivity in HER2-positive cells. These findings on CAP sensitivity provide the present invention with the ability to optimize CAP treatment to better overcome CAP resistance and thus prevent tumor recurrence.

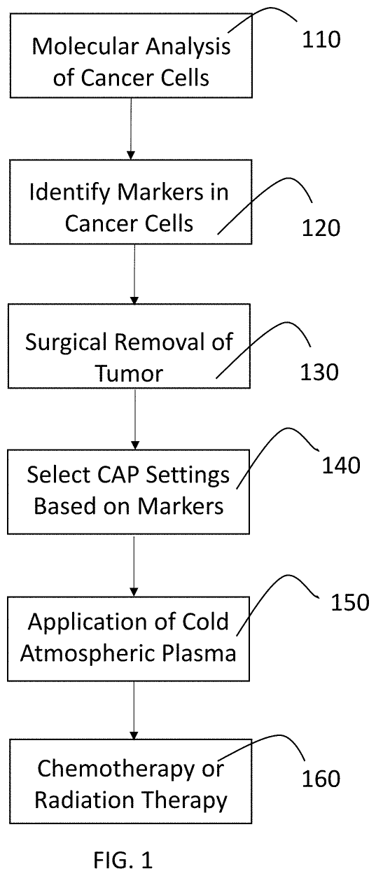

A method for treating breast cancer in accordance with a preferred embodiment of the present invention is shown in FIG. 1. Molecular analysis is performed on the target cancer cells (110) to identify markers in the cancer cells (120). The cancerous tumor is surgically removed (130). Settings for performing cold atmospheric plasma treatment are selected by a processor or CPU in the cold plasma generator based upon the markers identified in the molecular analysis (140). The margins surrounding the area where the cancerous tumor was removed are then treated with cold atmospheric plasma at the selected settings (150). A specific chemotherapy or radiation therapy is given to the patient (160) before, during or after the application of cold atmospheric plasma to reduce the cancer cells' resistance to cold atmospheric plasma therapy.

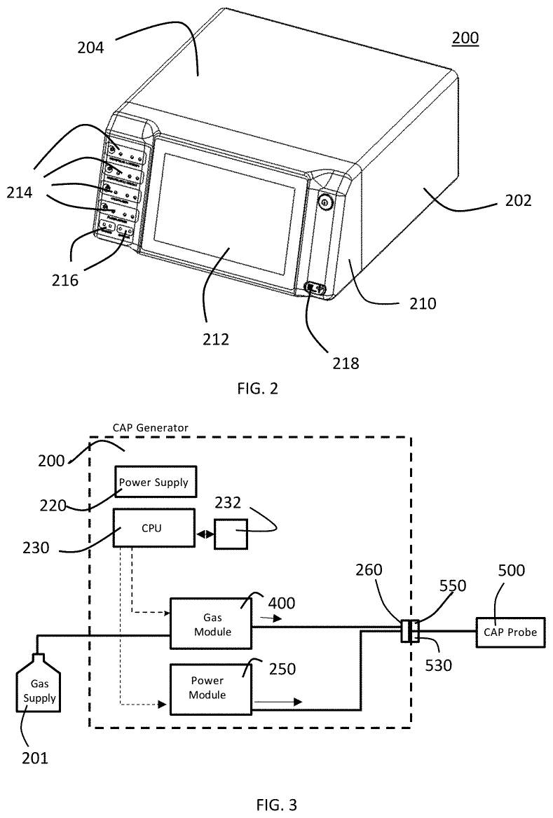

A preferred embodiment of a CAP enabled generator is described with reference to the drawings. A gas-enhanced electrosurgical generator 200 in accordance with a preferred embodiment of the present invention is shown in FIGS. 2 and 3. The gas-enhanced generator has a housing 202 made of a sturdy material such as plastic or metal similar to materials used for housings of conventional electrosurgical generators. The housing 202 has a removable cover 204. The housing 202 and cover 204 have means, such as screws, tongue and groove, or other structure for removably securing the cover to the housing. The cover 204 may comprise just the top of the housing or multiple sides, such as the top, right side and left side, of the housing 202. The housing 202 may have a plurality of feet or legs (not shown) attached to the bottom of the housing. The bottom of the housing 202 may have a plurality of vents (not shown) for venting from the interior of the gas-enhanced generator.

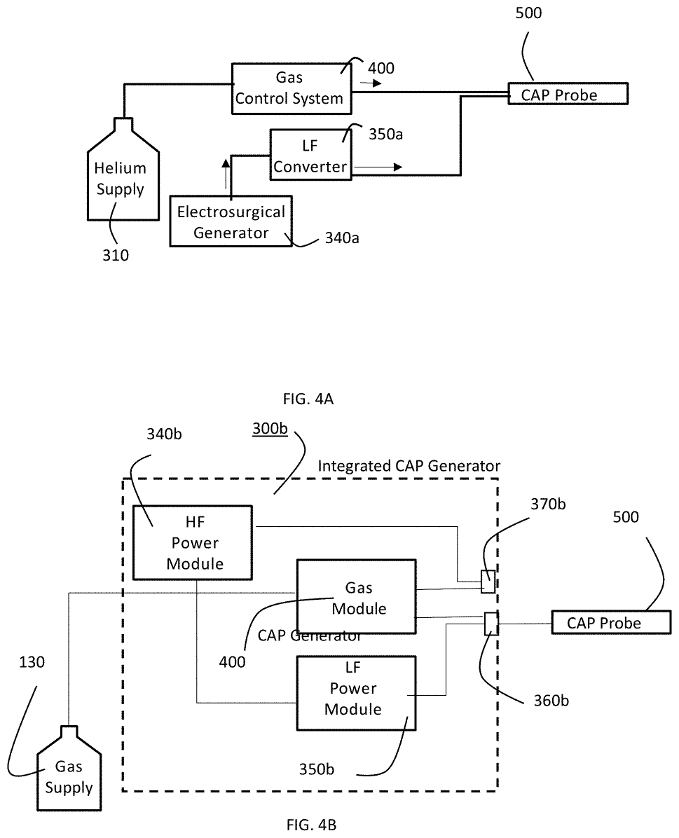

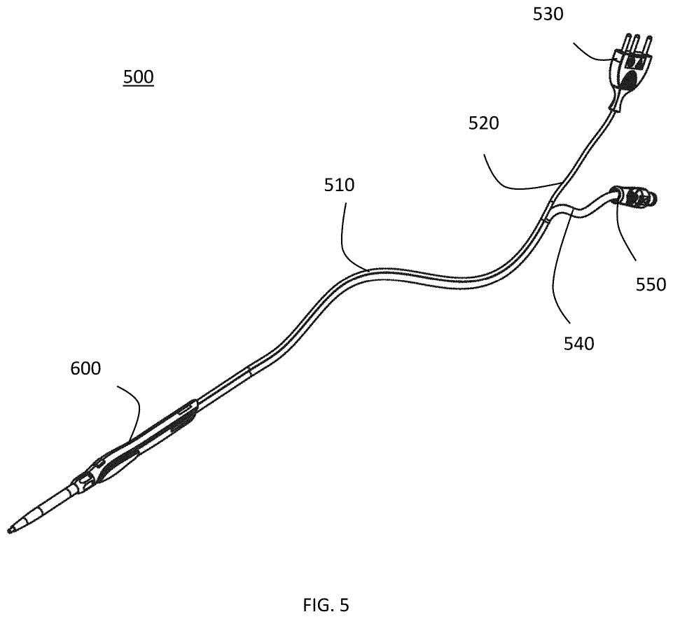

A generator housing front panel 210 is connected to the housing 202. On the face front panel 210 there is a touchscreen display 212 and there may be one or a plurality of connectors 214 for connecting various accessories to the generator 200. For a cold atmospheric plasma generator such as is shown in FIG. 3, for example, there is a connector 260 for connecting a cold atmospheric probe 500. An integrated multi-function electrosurgical generator, such as is shown in FIG. 4B the plurality of connectors may include an argon plasma probe, a hybrid plasma probe, a cold atmospheric plasma probe, or any other electrosurgical attachment. The face of the front panel 210 is at an angle other than 90 degrees with respect to the top and bottom of the housing to provide for easier viewing and use of the touch screen display 212 by a user.

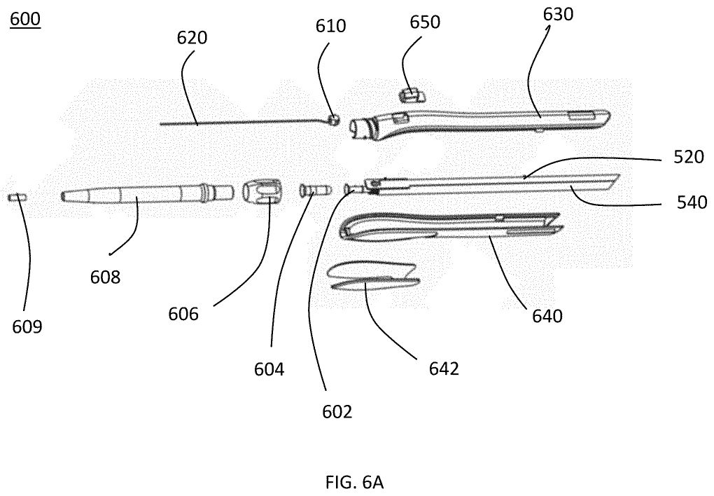

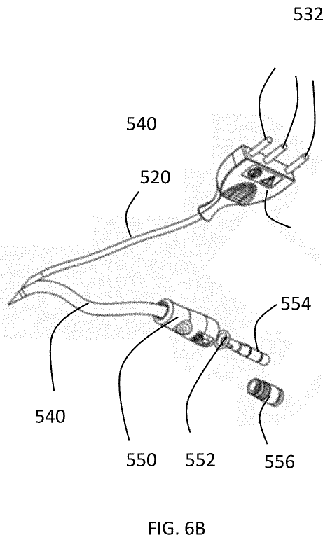

As shown in FIG. 3, an exemplary cold atmospheric plasma (CAP) generator 200 has a power supply 220, a CPU (or processor or FPGA) 230 and a memory or storage 232. The system further has a display 212 (FIG. 2), which may be the display of a tablet computer. The CPU 230 controls the system and receives input from a user through a graphical user interface displayed on display 212. The CAP generator further has a gas control module 400 connected to a source 201 of a CAP carrier gas such as helium. The gas control module 400 may be, for example, of the design described in International patent Application No. WO 2018/191265, which is hereby incorporated by reference. The CAP generator 200 further has a power module 250 for generating low frequency radio frequency (RF) energy, such as is described in U.S. Pat. No. 9,999,462, which is hereby incorporated by reference in its entirety. The power module 250 contains conventional electronics and/or transformers such as are known to provide RF power in electrosurgical generators. The power module 250 operates with a frequency between 10-200 kHz, which is referred to herein as a “low frequency,” and output peak voltage from 3 kV to 6 kV and preferably at a frequency near (within 20%) of 40 Hz, 100 Hz or 200 Hz. The gas module 400 and power module 250 are connected to connector 260 that allows for attachment of a CAP applicator 500 (as shown in FIGS. 5, 6A and 6B) to be connected to the generator 200 via a connector having an electrical connector 530 and gas connector 550.

As shown in FIG. 4B, other arrangements for delivery of the carrier gas and the electrical energy may be used with the invention. In FIG. 4B, an integrated CAP generator 300b is connected to a source 310 of a carrier gas (helium in this example), which is provided to a gas control system 400, which supplies the gas at a controlled flow rate to CAP applicator 500. A high frequency (HF) power module 340b supplies high frequency (HF) energy to a low frequency power module (converter) 350b, which outputs electrical energy having a frequency in the range of 10 kHz to 200 kHz and an output voltage in the range of 3 kV to 6 Kv. This type of integrated generator will have both a CAP connector 360b for connecting a CAP applicator or other CAP accessory and a connector 370b for attaching HF electrosurgical attachments such as an argon plasma or hybrid plasma probe (not shown).

Another embodiment, shown in FIG. 4A, has a carrier gas source 310 connected to a conventional gas control system 370, which in turn is connected to the CAP applicator 500, and a conventional electrosurgical generator 340 connected to a low frequency (LF) converter 350a, which is then connected to the CAP probe 500.

In the above-disclosed embodiment, a cold atmospheric plasma below 35° C. is produced. When applied to the tissue surrounding the surgical area, the cold atmospheric plasma induces metabolic suppression in only the tumor cells and enhances the response to the drugs that are injected into the patient.

The cold plasma applicator 500 may be in a form such as is disclosed in U.S. Pat. No. 10,405,913 and shown in FIGS. 5, 6A and 6B. A hand piece assembly 600 has a top side piece 630 and a bottom side piece 640. A control button 650 extends from the interior of the hand piece through an opening in the top side piece 630. Within the hand piece 600 is body connector funnel 602, PCB board 608, electrical wiring 520 and hose tubing (PVC medical grade) 540. The wiring 520 and hose tubing 540 are connected to one another to form a wire and tubing bundle 510. A grip over mold 642 extends over the bottom piece portion 640. In other embodiments, a grip may be attached to the bottom piece 640 in other manners. A probe or scalpel assembly is attached to the end of the hand piece. The probe assembly has non-bendable telescoping tubing 606, a ceramic tip 609, a column nut or collet 606 and body connector tubing 604. The hose tubing 540 extends out of the proximal end of the hand piece to a body gas connector 550, which has an O-ring 552, gas connector core 554 and gas connector tip 556 for connecting to a connector on a gas-enhanced electrosurgical generator. The printed circuit board 608 connects to electrical wiring 520 which leads to electrical connector 530 having electrical pins 532. Inside the handpiece 600 is an electrode 620 and conductive connector 610. There is a control button 650 for controlling the application of electrical energy.

Through experiments such as are described below, most-effective CAP setting can be determined with respect to various genetic markers in cancer cells. Using results of such experiments, a database of most-effective settings and corresponding molecular makers can be generated and stored in the memory 232 of the CAP generator 200 or may be stored elsewhere and accessed by the CPU 230 in the generator. The graphical user interface on the touchscreen display 212 then may be used to select or enter particular genetic markers to cause the CAP generator to automatically select the preferred settings for performing CAP on a particular line of target cancer cells. The database, of course, may include more complicated sets of data such as a type of chemotherapy or radiation therapy to be performed in conjunction with the application of CAP, for example, such that the CAP generator may select the appropriate CAP setting based upon the specific markers found in the molecular analysis of the cancer cells in combination with a type of other therapy being performed.

### Experiments

The present inventors evaluated the efficacy of the cold atmospheric plasma on various breast cancer cell lines based on ER, PR, and HER2 status. The human breast cancer cell lines that were studied include MCF-7, T-47D, SK-BR-3, BT-474, MDA-MB-231, Hs578T, and HCC1806. Receptor status of these cell lines are shown in Error! Reference source not found.

| Cell Line | ER (+/−) | PR (+/−) | HER2 (+/−) |
|---|---|---|---|
| MCF-7 | + | + | − |
| T-47D | + | + | − |
| SK-BR-3 | − | − | + |
| BT-474 | + | + | + |
| MDA-MB-231 | − | − | − |
| Hs578T | − | − | − |
| HCC1806 | − | − | − |

### Methods

### Cold Plasma Device

All experiments were performed at Jerome Canady Research Institute for Advanced Biological and Technological Sciences, Takoma Park, Md., USA, using a Cold Plasma Conversion System. The electrosurgical device consisted of the USMI SS-601 MCa high-frequency electrosurgical generator (USMI, Takoma Park, Md., USA) integrated with a USMI Canady Cold Plasma Conversion Unit and connected to a Canady Helios Cold Plasma™ Scalpel. The conversion unit has three connectors: a gas connector (to a helium tank), and electrical connector (to the generator), and an electro-gas connector (to the scalpel). The conversion unit also features a high voltage transformer that up-converts voltage up to 4 kV, down-converts frequency to less than 300 kHz, and down-converts power less than 40 W. Additional details and schematics on plasma generation are described in Cheng, X., et al., Treatment of Triple-Negative Breast Cancer Cells with the Canady Cold Plasma Conversion System: Preliminary Results. Plasma, 2018. 1(1): p. 218-228.

The helium flow rate was set to a constant 3 L/min and the power was set to 80, 100 and 120 P. The plasma scalpel tip was placed 1.5 cm above the surface of the cell media and remained unmoved for the duration of the treatment. The CAP treatment was performed in a laminar flow tissue culture hood, Purifier Logic+ Class II, Type A2 Biosafety Cabinet (Labconco, Kansas City, Mo., USA) at room temperature.

### Cell Culture

Human breast cancer cell lines T-47D, SK-BR-3, and BT-474, were purchased from ATCC (Manassas, Va., USA). MCF-7, MDA-MB-231, Hs578T, and HCC1806 were generously donated by Professor Kanaan's laboratory at Howard University. All cell lines except SK-BR-3 were cultured in Roswell Park Memorial Institute (RPMI) 1640 Medium supplemented with 10% fetal bovine serum (Sigma-Aldrich, St. Louis, Mo., USA) and 1% Pen Strep (Thermo Fisher Scientific, Waltham, Mass., USA) in a 37° C. and 5% CO₂ humidified incubator (Thermo Fisher Scientific, Waltham, Mass., USA). The exceptions for culture conditions include T-47D, which was additionally supplemented with 0.5 mg/mL insulin. SK-BR-3 was cultured in McCoy's 5A Medium. When cells reached approximately 80% confluence, cells were seeded at a concentration of 10⁵ cells/well into 12-well plates (USA Scientific, Ocala, Fla., USA) with a 1 mL media volume per well for cell viability assays.

### Cell Viability Assay

Thiazolyl blue tetrazolium bromide (MTT) assay was performed on the cells 48 h after plasma treatment following the manufacturer's protocol with all MTT assay reagents purchased from Sigma-Aldrich (St. Louis, Mo., USA). The absorbance of the dissolved compound was measured by BioTek Synergy HTX (Winooski, Vt., USA) microplate reader at 570 nm.

### Statistics

All viability assays were repeated 3 times with 2 replicates each. Data was plotted by Microsoft Excel 2016 as the mean±standard error of the mean. A student t-test or a one-way analysis of variance (ANOVA) was used to check statistical significance where applicable. The differences were considered statistically significant for * p≤0.05. A one-way multivariate analysis of variance (MANOVA) followed by a Post-Hoc test was used to check statistical significance where applicable. The differences were considered statistically significant for **f≤0.0176 and for *** f≤0.0033.

### Results

### Reduction of Viability by CAP in Breast Cancer Cell Lines

Various operational parameters of the cold atmospheric plasma system were tested to determine the CAP dosage necessary to significantly reduce viability of each breast cancer cell line. A flow rate of 3 L/min was selected for all experimental conditions with power settings of 80, 100, and 120 P. Treatment duration ranged from 1-6 min to reduce viability by at least 90%. MTT assays were performed to assess viability 48 hours post-CAP treatment.

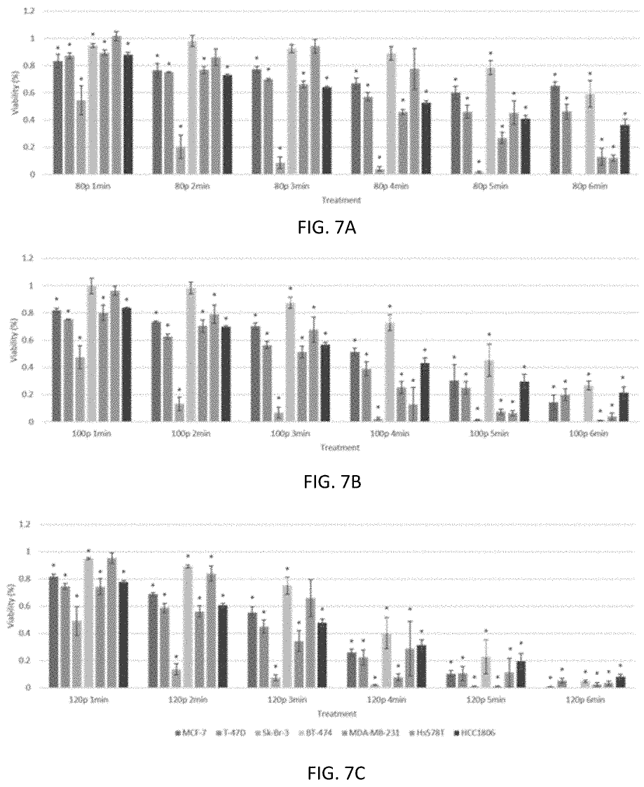

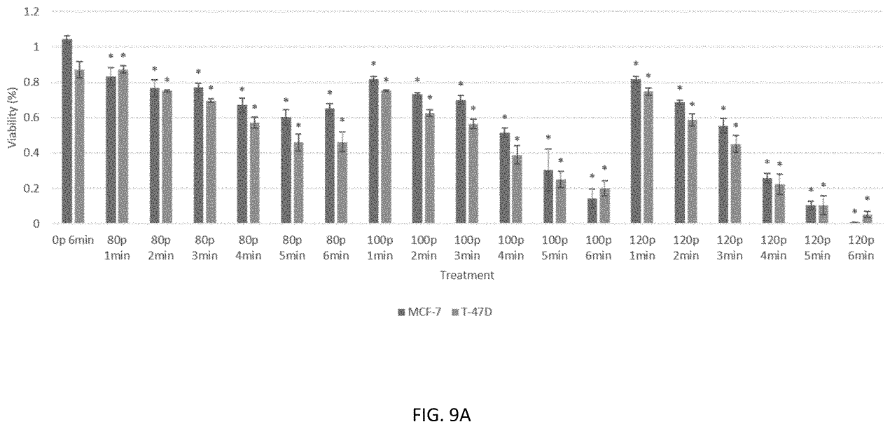

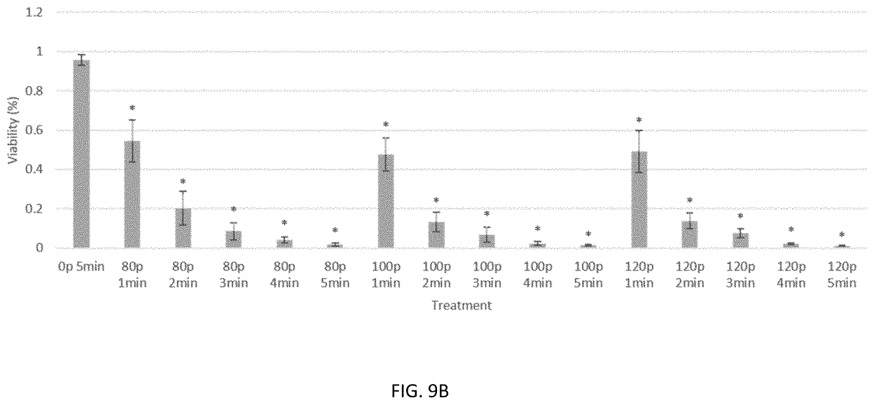

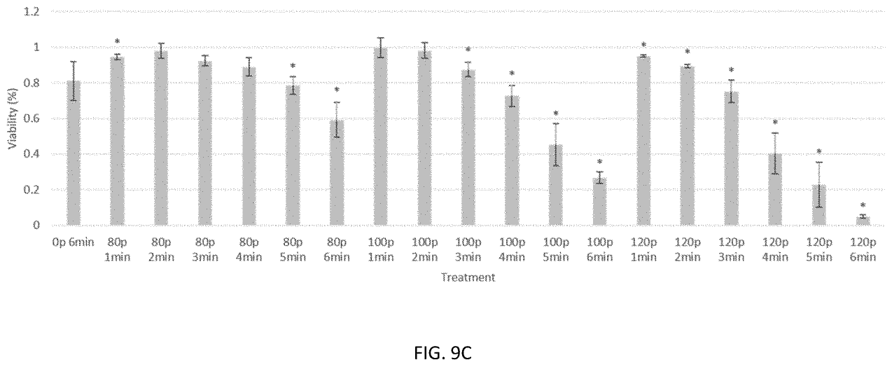

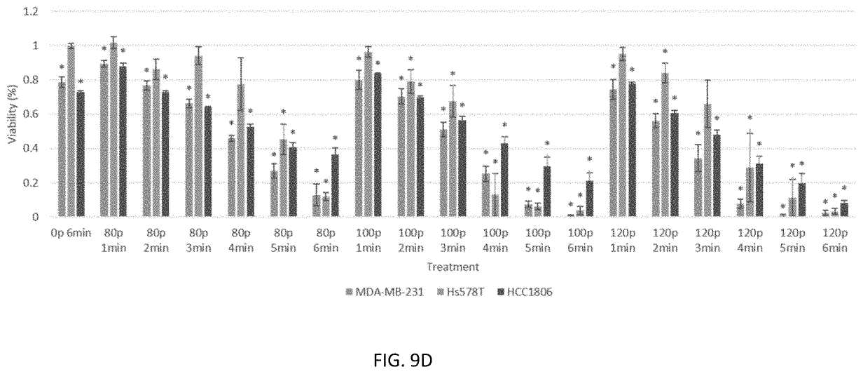

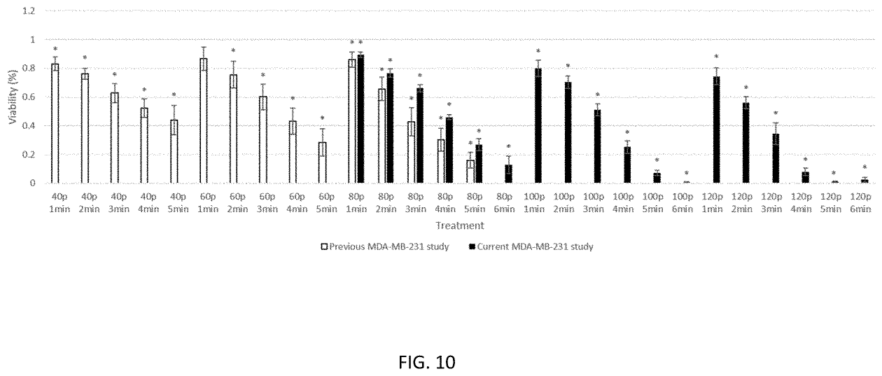

FIGS. 7A-7C demonstrate that CAP produced by the cold atmospheric plasma system had a clear time and dose dependent effect on the reduction of viability across all seven breast cancer cell lines that were tested. Viability data categorized by cell line subtypes can be found in FIGS. 9A-9D. Helium (Op), for the maximum treatment duration, minimally reduced cell viability (FIG. 9D) with no significant effect on MDA-MB-231 and HCC1806 viability (FIG. 9D). Increasing power and treatment duration from 40-80 P for 1-5 mins to 80-120 P for 1-6 minutes yielded a greater reduction of viability in MDA-MB-231 (FIG. 10). Treatment duration and power variability significantly reduced breast cancer cell viability, as shown in FIGS. 11-18B (** f_{(2,2)}≤0.0176, *** f_{(5,14)}≤0.0033). Ultimately, a 92-99% decrease in breast cancer viability was achievable across all breast cancer cell lines 48 h after 120 P at 5 or 6 min of CAP treatment (p≤0.05) (FIGS. 7A-7C).

The viabilities of both the ER+/PR+/HER2− cell lines, MCF-7 and T-47D were reduced to approximately 1% (p≤0.0001) and 5% (p≤0.0001), respectively, after given the highest CAP dose of 120 P for 6 min (FIGS. 7A-7C). For SK-BR-3, it was unnecessary to extend CAP treatment duration past 5 min since viability was already reduced by 99% after 5 min of CAP treatment at all tested powers (p≤0.0001) (FIGS. 7A-7C). Even with 80 P 1 min of CAP treatment, the most minimal dose, SK-BR-3 viability was nearly halved (p≤0.015) in contrast to all other cell lines, excluding Hs578T, which saw a ≤20% reduction in viability (p≤0.03). Unlike ER−/PR−/HER2+ cells, TPBC cells, required stronger doses and longer treatment to reduce viability (FIGS. 7Error! Reference source not found.A-7C). Treatment of 5 min at 100 P was the minimum CAP dosage to halve BT-474 viability (p≤0.01) (FIGS. 7A-7C). The only dosage to decrease BT-474 viability by >80% was 120 P for 6 min, in which viability was reduced by 95% (p≤0.0001) (Error! Reference source not found.7A-7C). Viabilities were reduced by 92-98% (p≤0.0001) in all three TNBC cell lines with HCC1806 showing the greatest overall CAP resistance (FIGS. 7A-7C). Amongst all seven tested breast cancer cell lines varying in receptor status, our data demonstrated ER−/PR−/HER2+ cells to be the most CAP susceptible and TPBC cells to be the most CAP resistant (FIGS. 7A-7C).

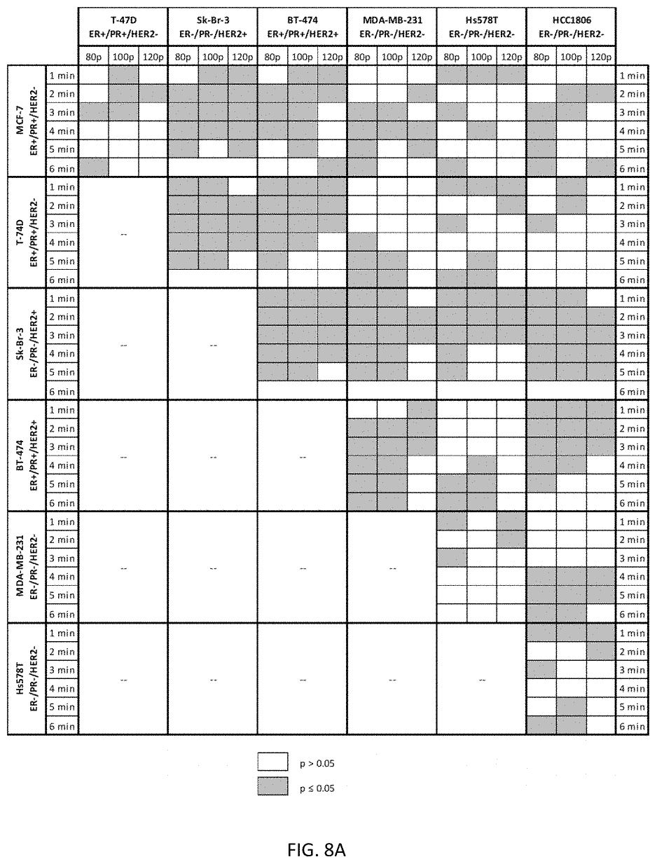

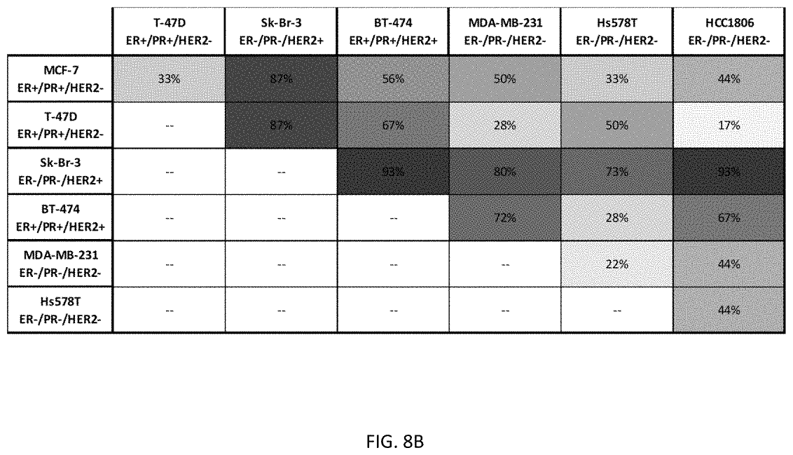

To evaluate whether receptor status was also significant factor in the reduction of viability by CAP treatment, the statistical significance of viability data was considered between cell lines across all treatment condition, displayed in FIG. 8A. There was a 33% difference between the two ER+/PR+/HER2− cell lines (p≤0.05) and a 22-44% difference between the three TNBC cell lines (p≤0.05) (FIG. 8B), suggesting breast cancer cells with identical receptor status have similar susceptibility to CAP. This is also further supported by the 17-50% difference between HER2-negative cell lines (p≤0.05). Compared to all other cell lines, the ER−/PR−/HER+ cell line was the most significantly different by 73-93% (p≤0.05) (FIG. 8FIGs. B), indicating ER−/PR−/HER2+ as a significant factor contributing to CAP susceptibility.

### Discussion

The purpose of these experiments was to determine the sensitivity of breast cancer cell lines to CAP treatment based on the receptor status. Identical receptor status showed the least difference in CAP sensitivity within the two ER+/PR+/HER2− cell lines (33%) and the three TNBC cell lines (22-44%) (p≤0.05) (FIG. 8B). HER2-negative status, irrespective of ER/PR status, also showed ≤50% difference in susceptibility when cell lines MCF-7 (ER+/PR+/HER2−), T-47D (ER+/PR+/HER2−), MDA-MB-231 (ER−/PR−/HER2−), Hs578T (ER−/PR−/HER2−), and HCC1806 (ER−/PR−/HER2−) are compared (p≤0.05) (FIG. 8B). Moreover, in the presence of HER2+ status, ER/PR status is significant in determining CAP sensitivity when Sk-Br-3 (ER−/PR−/HER2+) and BT-474 (ER+/PR+/HER2+) are compared (FIGS. 8A-8B). While 1 min of CAP treatment, regardless of power, nearly halved CAP-sensitive Sk-Br-3 viability (p≤0.015), it required 5 min 100 P of CAP to produce the same result in CAP-resistant BT-474 cells (p≤0.01) (FIGS. 7A-7C). Although significant resistance of TPBC to CAP treatment was demonstrated, the viability of TPBC cell viability was significantly reduced by 95% (p≤0.0001).

FIG. 9A is a bar graph showing the reduction of cell viability of ER+/PR+/HER2− cell lines 48 h after CAP treatment compared to no treatment controls. CAP treatment significantly reduces viability of MCF-7 and T-47D at all tested doses. * p≤0.05.

FIG. 9B is a bar graph showing the reduction of cell viability of an ER−/PR−/HER2+ cell line 48 h after CAP treatment compared to no treatment controls. CAP treatment significantly reduces viability of Sk-Br-3 at all tested doses. * p≤0.05.

FIG. 9C is a bar graph showing the reduction of cell viability of an ER+/PR+/HER2+ cell line 48 h after CAP treatment compared to no treatment controls. CAP treatment significantly reduces viability of BT-474 at nearly all tested doses. * p≤0.05.

FIG. 9D is a bar graph showing the reduction of cell viability of ER−/PR−/HER2-cell lines 48 h after CAP treatment compared to no treatment controls. CAP treatment significantly reduces viability of MDA-MB-231 and HCC1806 at all tested doses. CAP treatment significantly reduces viability of Hs578T at nearly all tested doses. * p≤0.05.

FIG. 10 is a bar graph further illustrating reduction of MDA-MB-231 cell viability by CCPCS 48 h after CAP treatment compared to no treatment controls. Our current experiment, which tested relatively higher CAP doses (80, 100, 120 P at 1-6 min), yielded a greater reduction of MDA-MB-231 viability 48 h after CAP treatment compared to that which involved relatively lower CAP doses (40, 60, 80 P at 1-5 min). The difference of 80 P viability between the two sets of data could be resulted by the different origin or passage of the cells, or the slight impedance change of the CAP device. The decreasing trend with the increase of treatment time remains the same regardless of the absolute value. * p≤0.05.

FIGS. 11A-11B are bar graphs showing (FIG. 11A) treatment duration and (FIG. 11B) power as factors in the reduction of MCF-7 viability compared to no treatment controls. At 100 P, increasing the treatment duration from 1 and 3 min to 4 min and 6 min, respectively, significantly reduced viability (***f_{(5,14)}≤0.003). At 120 P, increasing the treatment duration from 1-3 min to 4 min or longer made a significant difference in viability (***f_{(5,14)}≤0.0025). At 4, 5, and 6 min, increasing the power from 80 P to 120 P significantly reduced viability (**f_{(2,2)}≤0.0025). Brackets indicate significant difference between two treatment conditions. ** f_{(2,2)}≤0.0176. *** f_{(5,14)}≤0.0033. #=helium control was not considered for statistical analysis.

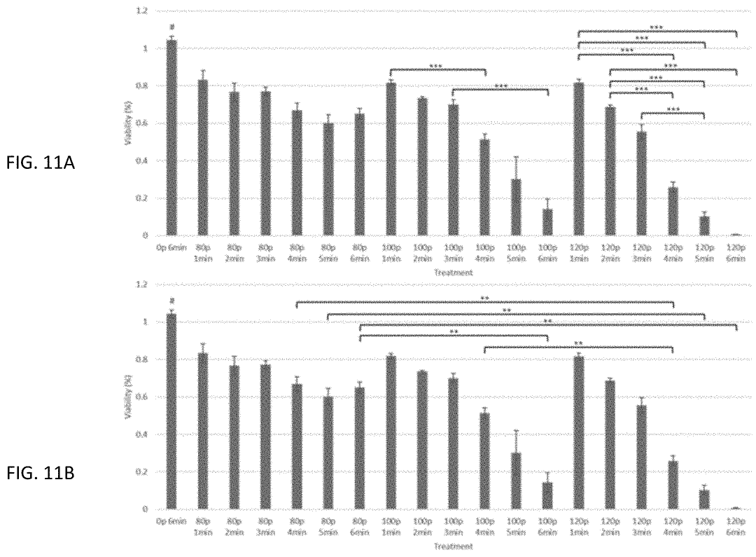

FIGS. 12A and 12B are bar graphs showing (FIG. 12A) treatment duration and (FIG. 12B) power as factors in the reduction of T-47D viability compared to no treatment controls. At 120p, increasing CAP treatment duration from 1 min to at least 4 min significantly reduced viability (***f_{(5,14)}≤0.0033). At 1, 4, and 5 min, increasing the power from 80 P to 120 P significantly reduced viability (**f_{(2,2)}≤0.015). Brackets indicate significant difference between two treatment conditions. ** f_{(2,2)}≤0.0176. *** f_{(5,14)}≤0.0033. #=helium control was not considered for statistical analysis.

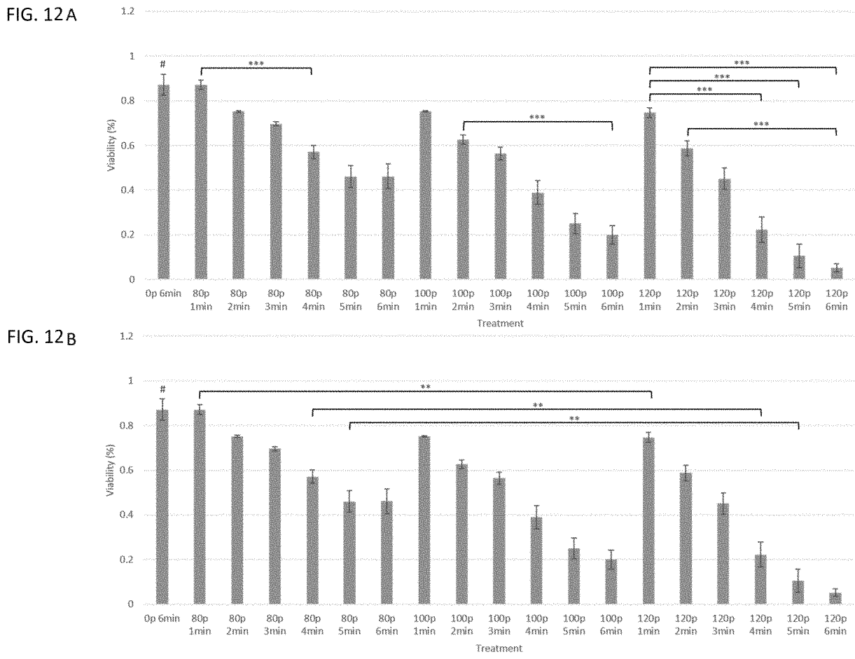

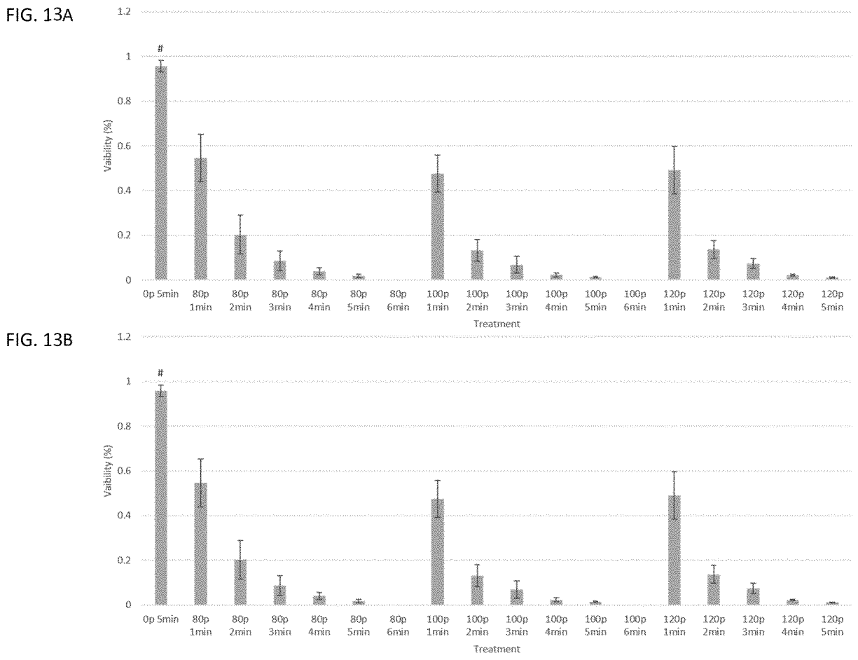

FIGS. 13A and 13B are bar graphs showing (FIG. 13A) treatment duration and (FIG. 13B) power as factors in the reduction of SK-BR-3 viability compared to no treatment controls. CAP treatment reduces viability regardless of treatment duration and power. Brackets indicate significant difference between two treatment conditions. ** f_{(2,2)}≤0.0176. *** f_{(5,14)}≤0.0033. #=helium control was not considered for statistical analysis.

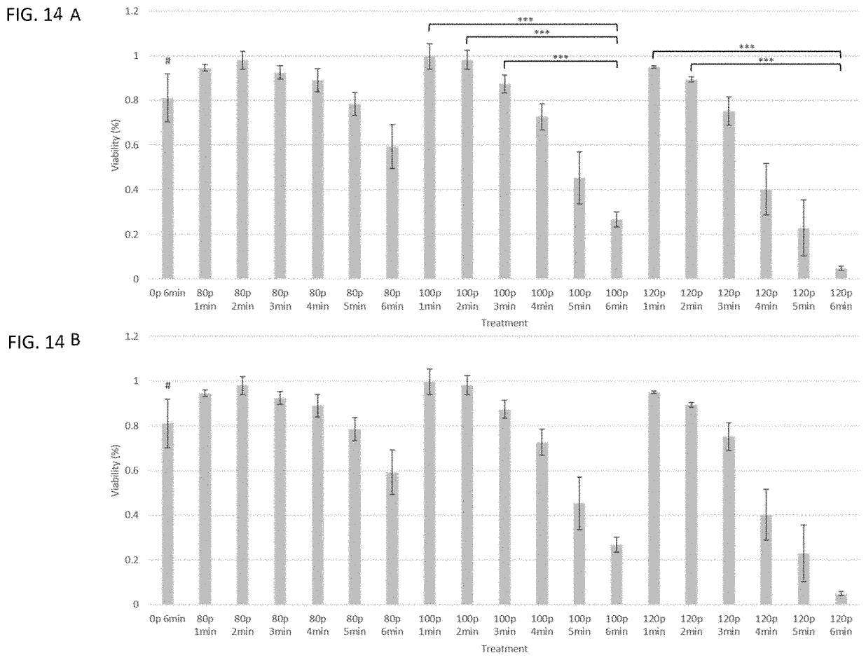

FIGS. 14A and 14B are bar graphs showing (FIG. 14A) treatment duration and (FIG. 14B) power as factors in the reduction of BT-474 viability compared to no treatment controls. At 100 P, increasing treatment duration from 1, 2, and 3 min to 6 min significantly reduces viability (***f_{(5,14)}≤0.002). At 120 P, increasing treatment duration from 1 and 2 min to 5 min significantly reduces viability (***f_{(5,14)}≤0.000004). CAP reduces viability regardless of power. Brackets indicate significant difference between two treatment conditions. ** f_{(2,2)}≤0.0176. *** f_{(5,14)}≤0.0033. #=helium was control not considered for statistical analysis.

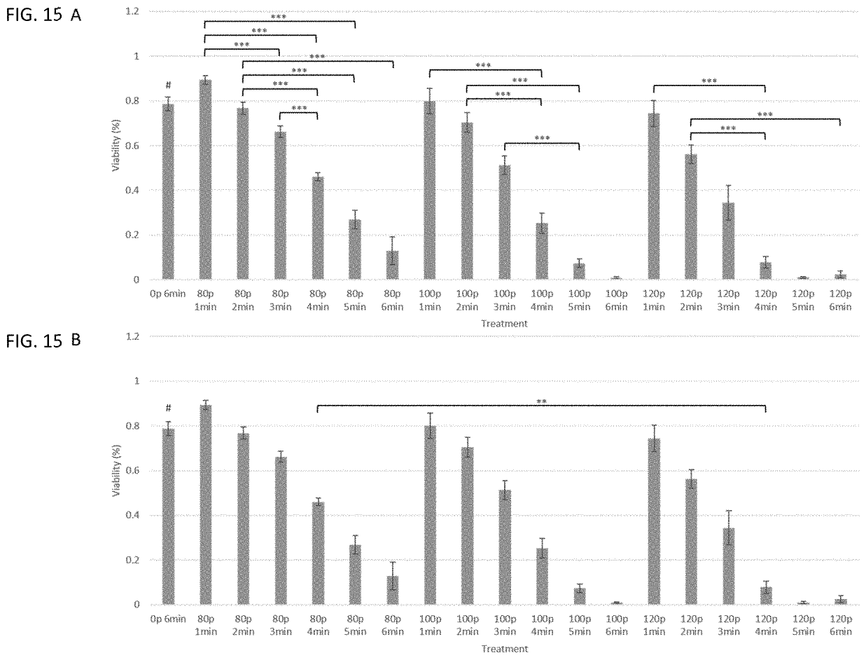

FIGS. 15A and 15B are bar graphs showing (FIG. 15A) treatment duration and (FIG. 15B) power as factors in the reduction of MDA-MB-231 viability compared to no treatment controls. At 80, 100, and 120 P, increasing treatment duration from 1, 2, or 3 min by at least 2 min, significantly reduces viability (***f_{(5,14)}≤0.003). At 4 min, increasing the power from 80 P to 120 P significantly reduces viability (**f_{(2,2)}≤0.0015). Brackets indicate significant difference between two treatment conditions. ** f_{(2,2)}≤0.0176. *** f_{(5,14)}≤0.0033. #=helium control was not considered for statistical analysis.

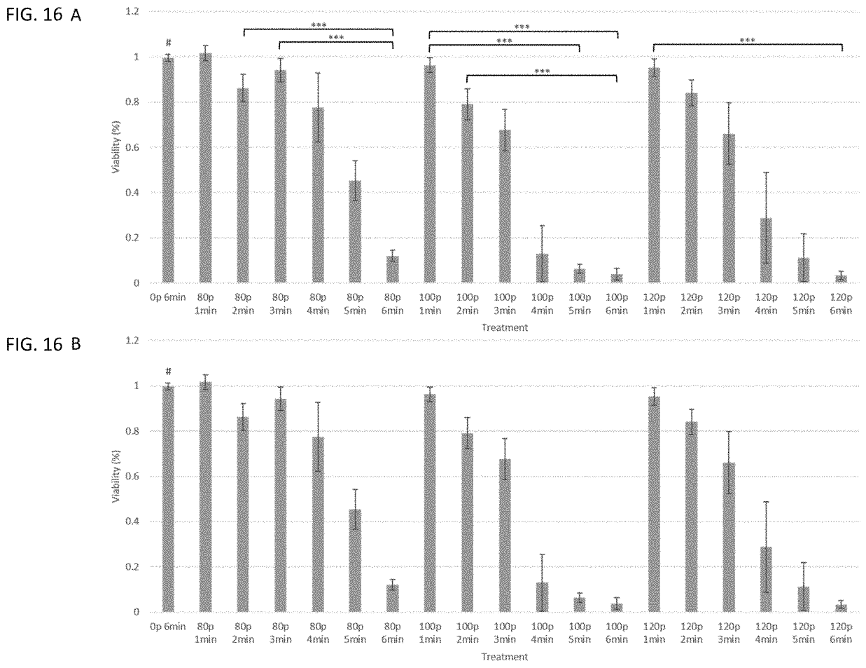

FIGS. 16A and 16B are bar graphs showing (FIG. 16A) treatment duration and (FIG. 16B) power as factors in the reduction of Hs578T viability compared to no treatment controls. At 80, 100, and 120 P, increasing treatment duration from 1, 2 or 3 min to 5 or 6 min significantly reduces viability (***f_{(5,14)}≤0.0015). CAP reduces viability regardless of power. ** f_{(2,2)}≤0.0176. *** f_{(5,14)}≤0.0033. #=helium control was not considered for statistical analysis.

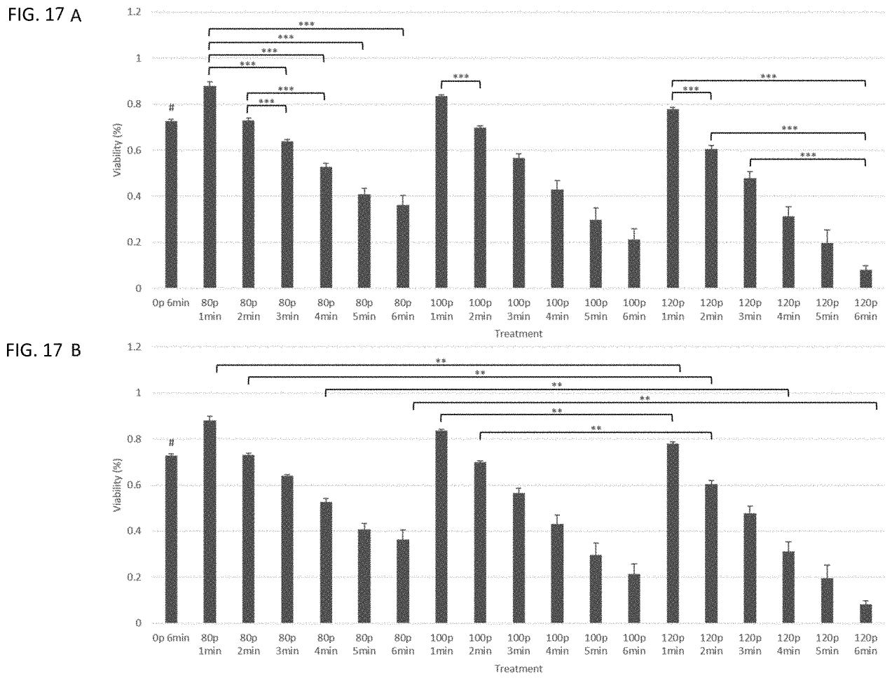

FIGS. 17A and 17B are bar graphs showing (FIG. 17A) treatment duration and (FIG. 17B) power as factors in the reduction of HCC1806 viability compared to no treatment controls. At 80 P, increasing treatment duration from 1 min by at least 2 min, significantly reduces viability (***f_{(5,14)}≤0.0015). At 120 P, increasing treatment duration from 1-3 min to 6 min significantly reduces viability (***f_{(5,14)}≤0.0025). Increasing power from 80 and 100 P to 120 P significantly reduces viability (**f_{(2,2)}≤0.015). Brackets indicate significant difference between two treatment conditions. ** f_{(2,2)}≤0.0176. *** f_{(5,14)}≤0.0033. #=helium control was not considered for statistical analysis.

This data suggests that ER/PR status is most important determining factor in CAP susceptibility for HER2+ breast cancer cells. Testing additional TPBC and ER−/PR−/HER2+ cell lines in future studies could further supplement our findings on CAP sensitivity. Nonetheless, the present data suggests molecular profile should be considered when determining the optimal CAP dosage for the treatment of breast cancer, especially in HER2+ breast cancer. Potentially, adjuvant and neoadjuvant trastuzumab or hormonal therapy alongside CAP treatment could improve the chance of overall survival in HER2+ breast cancer patients. The molecular pathway that determines the susceptibility of HER2+ cells to CAP treatment warrants further investigation. An insight on CAP mechanism will give us a better understanding on how to optimize CAP treatment to better overcome CAP resistance.

The data indicates that differential regulation of apoptotic genes is a major contributor to CAP selectivity. The importance of apoptotic malfunction in the TNBC prognosis is well documented in several studies. Nedeljkovic, M. and A. Damjanovic, Mechanisms of Chemotherapy Resistance in Triple-Negative Breast Cancer-How We Can Rise to the Challenge. Cells, 2019. 8(9). Poor prognosis in TNBC is attributed to pro-survival factors, such as B-cell lymphoma 2 (Bcl-2) (Inao, T., et al., Bcl-2 inhibition sensitizes triple-negative human breast cancer cells to doxorubicin. Oncotarget, 2018. 9(39): p. 25545-25556) and myeloid cell leukemia 1 (Mcl-1). Additionally, TRAIL receptors also contribute to apoptosis dysregulation, however targeted therapies against TRAIL and several DRs have failed to improve patient outcomes. Lemke, J., et al., Getting TRAIL back on track for cancer therapy. Cell Death Differ, 2014. 21(9): p. 1350-64. By identifying and targeting molecular markers responsible for CAP resistance, a greater reduction of viability by smaller CAP dosages can be achieved.

Different cell media may influence CAP sensitivity. In this experiment, SK-BR-3 was the only cell line to be cultured in McCoy 5A media as opposed to RPMI 1640 (all of which was in accordance with ATCC recommendations). A study done jointly with The George Washington University, investigated the interaction between CAP-generated effective species and amino acids present in the media. It was concluded that cysteine and tryptophan consumed the most CAP-generated effective species, thus weakening the anti-tumor ability of CAP on cells. See, Yan, D., et al., Principles of using Cold Atmospheric Plasma Stimulated Media for Cancer Treatment. Sci Rep, 2015. 5: p. 18339. However, when glioblastoma and breast cancer cells were cultured in the same type of media, glioblastoma cells consumed CAP-generated effective species at a faster rate than breast cancer cells, suggesting distinct expression of extracellular proteins dependent on cell line. Gene profiling reveals that oxidative stress-related genes cause preferential uptake of reactive oxygen and nitrogen species by CAP susceptible cells.

Promising cancer therapeutic potential of the cold atmospheric plasma system has been demonstrated. Additionally, CAP treatment alongside chemotherapy can further improve breast cancer management. Furthermore, the present findings on CAP sensitivity will be the foundation in the development of customized CAP-based therapy regiments for various breast cancer subtypes.

There is a present demand for a therapy that effectively treats all breast cancer subtypes regardless of receptor status since effectiveness of current endocrine and HER2-targeted therapies are dependent on receptor status. Liedtke, C., et al., Response to neoadjuvant therapy and long-term survival in patients with triple-negative breast cancer. Journal of Clinical Oncology, 2008. 26(8): p. 1275-81; Wahba, H. A. and H. A. El-Hadaad, Current approaches in treatment of triple-negative breast cancer. Cancer Biol Med, 2015. 12(2): p. 106-16. To this end, the cold atmospheric plasma system can be offered as a solution after demonstrating its ability to reduce breast cancer viability by 92-99% (p≤0.05) regardless of receptor status. The present experiments revealed that molecular profiling when selecting appropriate CAP dosages is beneficial especially in HER2-positive breast cancers in which ER/PR status is a significant factor in determining CAP sensitivity.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

What is claimed is:

1. A method for treatment of breast cancer comprising:
performing a molecular analysis of target breast cancer cells;
identifying markers associated with said target breast cancer cells based on said performed molecular analysis;
selecting on a graphical user interface of a cold atmospheric plasma generator said identified markers associated with said target breast cancer cells;
selecting with a processor in said cold atmospheric plasma generator preferred cold atmospheric plasma settings associated with said identified markers in a database stored in a storage in said cold atmospheric plasma generator; and
applying cold atmospheric plasma with said cold atmospheric plasma generator at said selected cold atmospheric pressure settings to said target breast cancer cells.

2. A method for treatment of breast cancer according to claim 1 wherein said settings include time and power.

3. A method for treatment of breast cancer according to claim 1, further comprising treating said target breast cancer cells with one of chemotherapy and radiation therapy to reduce resistance of the target cancer cells' resistance to cold atmospheric plasma treatment.
